(19) United States
(12) Patent Application Publication
(10) Pub. No.: US 2009/0136450 A1
(43) Pub. Date: May 28, 2009

(54) THERAPY FOR NEUROLOGICAL DISEASES

(75) Inventors: Ilya Chumakov, Vaux-le Penil (FR); Daniel Cohen, Le Vesinet (FR); Fabio Macciardi, Paris (FR)

Correspondence Address: SALIWANCHIK LLOYD & SALIWANCHIK;A PROFESSIONAL ASSOCIATION, PO Box 142950, GAINESVILLE, FL, 32614, US

(73) Assignee: ARES TRADING S.A., Aubonne (CH)

(21) Appl. No.: 11/997,185

(22) PCT Filed: Jul. 31, 2006
(86) PCT No.: PCT/EP2006/064870

§ 371 Date: Dec. 18, 2008

Related U.S. Application Data
(60) Provisional application No. 60/720,579, filed on Sep. 26, 2005.

(30) Foreign Application Priority Data
Aug. 1, 2005 (EP) 05291640.0

(51) Int. Cl. A61K 38/21; A61K 31/506; A61P 25/00; G01N 33/68; C12Q 1/68; A61K 31/404

(52) U.S. Cl. 424/85.6; 514/252.18; 514/414; 424/85.4; 435/6; 435/86

(57) ABSTRACT

The present invention relates to compositions and methods for treating neurological diseases in a subject. More specifically, the invention relates to combination therapies for treating such diseases, using a c-kit inhibitor and a neuroactive compound. The invention may be used against a variety of demyelinating diseases, including multiple sclerosis, in any mammalian subject, particularly human subjects, and at various stages of disease progression.

The present invention relates to compositions and methods for treating neurological diseases and more particularly demyelinating diseases (such as multiple sclerosis) in a subject. More specifically, the invention relates to combination therapies for treating such diseases, using a c-kit inhibitor and a neuroactive compound. The invention may be used against a variety of demyelinating diseases, including multiple sclerosis, in any mammalian subject, particularly human subjects, and at various stages of disease progression.

### BACKGROUND OF THE INVENTION

Demyelinating diseases are a group of pathologies that involve abnormalities in myelin sheaths of the nervous system. Many congenital metabolic disorders affect the developing myelin sheath, mainly in the CNS, and demyelination is a feature of many neurological disorder.

The most known chronic inflammatory demyelinating disease of the central nervous system in humans is multiple sclerosis. The onset of multiple sclerosis (MS) typically occurs during ages 20 to 40. Women are affected approximately twice as often as men. Over time, MS may result in the accumulation of various neurological disabilities. Clinical disability in MS is presumed to be a result of repeated inflammatory injury with subsequent loss of myelin and axons, leading to tissue atrophy.

MS is manifested in physical symptoms (relapses and disability progression), central nervous system (CNS) inflammation, brain atrophy and cognitive impairment. Presenting symptoms include focal sensory deficits, focal weakness, visual problems, imbalance and fatigue. Sexual impairment and sphincter dysfunction may occur. Approximately half of the patients with MS may experience cognitive impairment or depression.

MS is now considered to be a multi-phasic disease, and periods of clinical quiescence (remissions) occur between exacerbations. Remissions vary in length and may last several years but are infrequently permanent.

Four courses of the disease are individualized: relapsing-remitting (RR), secondary progressive (SP), primary progressive (PP) and progressive relapsing (PR) multiple sclerosis. More than 80% of patients with MS initially display a RR course with clinical exacerbation of neurological symptoms, followed by a recovery that may or may not be complete (Lublin and Reingold, Neurology, 1996, 46:907-911).

During RRMS, accumulation of disability results from incomplete recovery from relapses. Approximately, half of the patients with RRMS switch to a progressive course, called SPMS, 10 years after the diseased onset. During the SP phase, worsening of disability results from the accumulation of residual symptoms after exarcerbation but also from insidious progression between exacerbations (Lublin and Reingold above). 10% of MS patients have PPMS which is characterized by insidious progression of the symptoms from the disease onset. Less than 5% of patients have PRMS and are often considered to have the same prognosis as PPMS. It is suggested that distinct pathogenic mechanisms may be involved in different patient sub-groups and have wide-ranging implications for disease classification (Lassmann et al., 2001, Trends Mol. Med., 7, 115-121; Lucchinetti et al., Curr. Opin. Neurol., 2001, 14, 259-269).

MS onset is defined by the occurrence of the first neurological symptoms of CNS dysfunction. Advances in cerebrospinal fluid (CSF) analysis and magnetic resonance imaging (MRI) have simplified the diagnostic process and facilitated early diagnostic (Noseworthy et al., The New England Journal of Medicine, 2000, 343, 13, 938-952). The International Panel on the Diagnosis of MS issued revised criteria facilitating the diagnosis of MS and including MRI together with clinical and para-clinical diagnostic methods (Mc Donald et al., 2001, Ann. Neurol., 50:121-127).

Molecules currently used for the treatment of multiple sclerosis and other demyelinating diseases essentially act against the symptoms of the diseases. Consequently, there is a strong need for alternative molecules or therapies that provide improved clinical benefits to patients.

### SUMMARY OF THE INVENTION

The present invention now discloses novel approaches to treatment of neurological diseases, including demyelinating diseases such as multiple sclerosis. The invention more specifically demonstrates that alterations in the c-kit gene are associated with the development of such diseases, and now proposes, for the first time, novel therapies that are more effective at treating patients suffering from a neurological disease, particularly a demyelinating disease.

An object of this invention resides in the use of a c-kit inhibitor for the manufacture of a medicament for treating neurological diseases and more particularly demyelinating diseases (such as multiple sclerosis). The invention also resides in a method for treating neurological diseases and more particularly demyelinating diseases (such as multiple sclerosis) in a subject in need thereof, the method comprising administering to the subject a c-kit inhibitor.

An object of this invention also resides in the use of a combination of a c-kit inhibitor and a neuroactive compound or treatment for the manufacture of a medicament for treating a neurological disease, particularly a demyelinating disease.

A further object of this invention relates to a method for treating a neurological disease, particularly a demyelinating disease in a subject in need thereof, the method comprising administering to the subject a combination of a c-kit inhibitor and a neuroactive compound.

An other object of this invention is a method of preparing a pharmaceutical treatment for treating a neurological disease, particularly a demyelinating disease in a subject, the method comprising providing a c-kit inhibitor and a neuroactive compound in a form suitable for administration to a subject.

A further object of this invention is a product comprising a c-kit inhibitor and a neuroactive compound as a combined preparation for simultaneous, separate or sequential use in the therapy of a neurological disease, particularly a demyelinating disease in a mammalian subject, preferably a human subject.

A further object of this invention relates to an improved method for treating a neurological disease, particularly a demyelinating disease in a subject receiving neuroactive compound therapy, the improvement comprising administering to said patient an effective amount of a c-kit inhibitor.

As will be discussed below, the c-kit inhibitor and neuroactive compound may be administered according to various schedules or protocols, including simultaneously, separately and/or sequentially. Furthermore, repeated administrations may be performed, depending on the disease, dosages and subject.

A further object of this invention is a composition comprising a c-kit inhibitor and a neuroactive compound, for simultaneous, separate or sequential administration.

In a preferred embodiment, the neuroactive compound is an interferon, even more preferably a beta-interferon, and/or the c-kit inhibitor is imatinib.

The invention may be used to treat various demyelinating diseases, and is particularly suited for the treatment of multiple sclerosis. The invention may be used in any mammalian subject, particularly human subjects, at various stages of disease progression. It is particularly suited for treating a subject having a susceptibility alteration in a c-kit gene or polypeptide.

In this regard, a further aspect of this invention is a method of detecting the presence of or predisposition to a neurological disease, particularly a demyelinating disease in a subject, the method comprising detecting in vitro or ex vivo the presence or a susceptibility alteration in a c-kit gene or polypeptide in a sample from the subject, the presence of such an alteration being indicative of the presence of or predisposition to a neurological disease, particularly a demyelinating disease in the subject.

The invention also relates to a method of assessing the response or responsiveness of a subject to a treatment of a neurological disease, particularly a demyelinating disease, the method comprising detecting in vitro or ex vivo the presence of a susceptibility alteration in a c-kit gene or polypeptide in a sample from the subject, the presence of such an alteration being indicative of a responder subject.

As will be disclosed further, the susceptibility alteration is typically a single nucleotide polymorphism (SNP), such as more preferably a single nucleotide polymorphism as listed in Tables 2 and 3. The susceptibility alteration is detected by sequencing, selective hybridisation and/or amplification.

### DETAILED DESCRIPTION OF THE INVENTION

The present invention relates to novel combination therapies for treating neurological diseases and more particularly demyelinating diseases (such as multiple sclerosis) in a subject, using a c-kit inhibitor and a neuroactive compound. The present invention originally stems from association studies conducted by the inventors on different MS populations, unexpectedly showing that the c-kit gene is associated with multiple sclerosis and related disorders and that a combined therapeutic approach using neuroactive compounds and c-kit inhibitors provides improved and complementary therapeutic effects in patients.

The present invention thus provides novel means and methods to the treatment of neurological diseases and more particularly demyelinating disorders such as multiple sclerosis. The invention leads to an effective treatment and/or to reduced side effects in subjects affected with such diseases.

### DEFINITIONS

The term “neurological disease” as used in the context of the present invention encompasses both “neuroinflammatory diseases” and “demyelinating diseases”.

The term “demyelinating diseases” as used in the context of the present invention, designates any disease involving abnormalities in myelin sheaths of the nervous system, in particular destruction of myelin. Many congenital metabolic disorders (e.g., phenylketonuria and otheraminoacidurias; Tay-Sachs, Niemann-Pick, and Gaucher's diseases; Hurler's syndrome; Krabbe's disease and other leukodystrophies) affect the developing myelin sheath, mainly in the CNS. Unless the biochemical defect can be corrected or compensated for, permanent, often widespread, neurological deficits result. Demyelination in later life is a feature of many neurological disorders; it can result from damage to nerves or myelin due to local injury, ischemia, toxic agents, or metabolic disorders. Extensive myelin loss is usually followed by axonal degeneration and often by cell body degeneration, both of which may be irreversible. Central demyelination (i.e., of the spinal cord, brain, or optic nerves) is the predominant finding in the primary demyelinating diseases, whose etiology is unknown. The most well known demyelinating disease is multiple sclerosis (see below and Background section). Further demyelinating diseases comprise: acute disseminated encephalomyelitis, which is characterized by perivascular CNS demyelination, and which can occur spontaneously but usually follows a viral infection or viral vaccination; acute inflammatory peripheral neuropathies that follow a viral vaccination or the Guillain-Barre syndrome, they affect only peripheral structures; adrenoleukodystrophy and adrenomyeloneuropathy, which are rare X-linked recessive metabolic disorders characterized by adrenal gland dysfunction and widespread demyelination of the nervous system; progressive multifocal leukoencephalopathy (PML), acute disseminated encephalomyelitis (ADEM), Leber's hereditary optic atrophy and related mitochondrial disorders, which are characterized primarily by bilateral loss of central vision, and which can resemble the optic neuritis in MS; HTLV-associated myelopathy, a slowly progressive spinal cord disease associated with infection by the human T-cell lymphotrophic virus, that is characterized by spastic weakness of both legs, and Pelizaeus-Merzbacher disease.

The term “multiple sclerosis” (“MS”) may be defined as in the DSM-IV classification (Diagnosis and Statistical Manual of Inflammatory CNS Disorders, Fourth Edition, American Psychiatric Association, Washington D.C., 1994).

The term “treat” or “treating” as used herein is meant to ameliorate, alleviate symptoms, eliminate the causation of the symptoms either on a temporary or permanent basis, or to prevent, delay or slow the appearance of symptoms of the named disorder or condition. The term “treatment” as used herein also encompasses the term “prevention of the disorder”, which is, e.g., manifested by delaying the onset of the symptoms of the disorder to a medically significant extent. Treatment of the disorder is, e.g., manifested by a decrease in the symptoms associated with the disorder or an amelioration of the reoccurrence of the symptoms of the disorder. The term “treatment” generally refers to any beneficial effect on progression of disease, including attenuation, reduction, decrease or diminishing of the pathological development after onset of disease. As indicated, the term “treatment” also includes prevention, which refers not only to a complete prevention of the disease or one or more symptoms of the disease, but also to any partial or substantial prevention, attenuation, reduction, decrease or diminishing of the effect before or at early onset of disease.

The term “combination therapy” indicates that several active agents are used in combination. Such term, however, does not require a unique formulation of the active agents, nor their simultaneous administration, but designates the fact that the active agents provide a combined therapeutic effect when both present in vivo.

A first aspect of this invention resides in the use of a combination of a c-kit inhibitor and a neuroactive compound or treatment for the manufacture of a medicament for treating a neurological disease, particularly a demyelinating disease, as well as a corresponding method.

Within the context of this invention, a c-kit inhibitor designates any compound or treatment that inhibits (e.g., reduces, suppresses, abolishes), permanently or transiently, the activity of a c-kit protein.

A c-kit inhibitor may inhibit the synthesis of a c-kit protein in a cell, e.g., the expression, maturation, translocation to the membrane, etc. The inhibitor is most preferably a compound that binds a c-kit protein, most preferably at the surface of a cell, and that prevents or inhibits the activation or activity of said protein, i.e. its tyrosine kinase activity.

A preferred class of “c-kit inhibitors” contemplated by the present invention includes compounds which show activity, e.g., in the c-kit enzyme assay as disclosed in Example 2. Preferred c-kit inhibitors as used in the present invention exhibit, in the above-described assay, an IC₅₀ value between 50 and 2500 nM, more preferably between 250 and 2000 nM, and most preferably between 500 and 1250 nM.

The c-kit inhibitor may be of various nature and type, such as a small drug, a peptide, an antibody (or a fragment or derivative thereof), a lipid, a nucleic acid, etc. Most preferably, the inhibitor is a small drug; a small molecule antagonist inhibiting kinase activity, which may, preferably, target the enzymatic active catalytic pocket (e.g. ATP pocket), or which may represent allosteric inhibitors; an intracellular or extracellular peptide inhibitor; an antibody; a soluble receptor (trap technology, trapping away its ligand SCF), a SCF mutant (binding to Kit, but devoid of activity); a nucleic acid targeting expression of Kit (and/or SCF), such as: antisense, shRNAi, RNAi, miRNA etc.; or an aptamer.

In a first, preferred embodiment, the c-kit inhibitor is imatinib mesylate (STI-571, Gleevec™, Novartis) or a derivative thereof. Inatinib, which is on the market, is 4-(4-methylpiperazine-1-ylmethyl)-N-[4-methyl-3-(4-pyridine-3-yl)pyrimidine-2-ylamino)phenyl]-benzamide of formula:

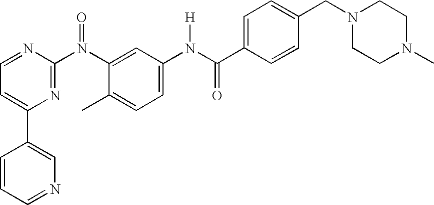

Derivatives include, generally, any pyrimidine derivative, more particularly an N-phenyl-2-pyrimidine-amine derivative, as described in WO03/002107, WO03/002109, WO03/072090, WO02080925 and EP 564 409. The c-kit inhibitors of interest encompass N-phenyl-2-pyrimidine-amine derivatives selected from the compounds corresponding to the following formula:

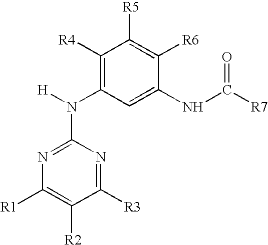

Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

Preferably, R7 is the following group:

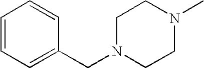

Among these compounds, the preferred are defined as follows:
R1 is a heterocyclic group, especially a pyridyl group,

### R2 and R3 are H,

R4 is a C1-C3 alkyl, especially a methyl group,

### R5 and R6 are H,

and R7 is a phenyl group bearing at least one substituent, which in turn possesses at least one basic site, such as an amino function, for example the group:

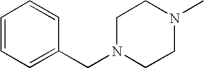

In a second preferred embodiment, the c-kit inhibitor is compound ZK-222584 (Novartis), which is in phase II trials, corresponding to the following formula:

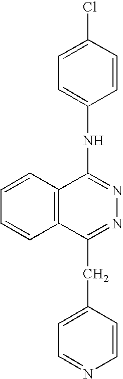

or a derivative thereof.

Other names are 1-Phthalazinamine, N-(4-chlorophenyl)-4-(4-pyridinylmethyl)-, butanedioate (1:1) (9CI); CGP 79787D; PTK 787; Vatalanib succinate.

More generally 4-Pyridylmethyl-phthalazine derivatives suitable as c-kit inhibitors are described in WO00/59509, WO01/10859 and, especially, in U.S. Pat. No. 6,258,812. Preferred 4-Pyridylmethyl-phthalazine derivatives of U.S. Pat. No. 6,258,812 have the following formula,

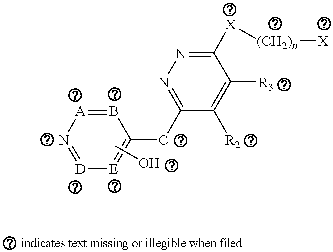

wherein r is 0 to 2, n is 0 to 2 μm is 0 to 4,
R1 and R2 (i) are lower alkyl, especially methyl, or
(ii) together form a bridge in subformula

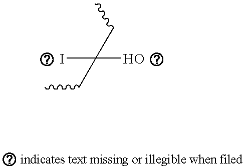

the binding being achieved via the two terminal carbon atoms, or
(iii) together form a bridge in subformula

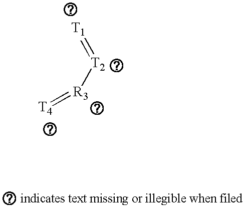

wherein one or two of the ring members T1, T2, T3 and T4 are nitrogen, and the others are in each case CH, and the binding is achieved via T1 and T4;
A, B, D, and E are, independently of one another, N or CH, with the stipulation that not more than 2 of these radicals are N;
G is lower alkylene, lower alkylene substituted by acyloxy or hydroxy, —CH2-O—, —CH2-S—, —CH₂—NH—, oxa (—O—), thia (—S—), or imino (—NH—);
Q is lower alkyl, especially methyl;
R is H or lower alkyl;
X is imino, oxa, or thia;
Y is aryl, pyridyl, or unsubstituted or substituted cycloalkyl; and
Z is mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl lower alkylthio, alkylphenylthio, phenylsulfinyl, phenyl-lower alkylsulfinyl, alkylphenylsulfinyl, phenylsulfonyl, phenyl-lower alkylsulfonyl, or alkylphenylsulfonyl, wherein—if more than 1 radical Z (m=.gtoreq.2) is present—the substituents Z are the same or different from one another; and wherein the bonds characterized, if present, by a wavy line are either single or double bonds;
or an N-oxide of the defined compound, wherein one or more N atoms carry an oxygen atom;
with the stipulation that, if Y is pyridyl or unsubstituted cycloalkyl, X is imino, and the remaining radicals are as defined, G is selected from the group comprising lower alkylene, —CH₂—O—, —CH₂—S—, oxa and thia.

Specific examples of such derivatives include the compounds named below: 1-(4-Chloroanilino)-4-(4-pyridylmethyl)phthalazine; 1-(3-Chloroanilino)-4-(4-pyridylmethyl)phthalazine; 1-Anilino-4-(4-pyridylmethyl)phthalazine; 1-Benzylamino-4-(4-pyridylmethyl)phthalazine; 1-(4-Methoxyanilino)-4-(4-pyridylmethyl)phthalazine; 1-(3-Benzyloxyanilino)-4-(4-pyridylmethyl)phthalazine; 1-(3-Methoxyanilino)-4-(4-pyridylmethyl)phthalazine; 1-(2-Methoxyanilino)-4-(4-pyridylmethyl)phthalazine; 1-(4-Trifluoromethylanilino)-4-(4-pyridy=methyl)phthalazine; 1-(4-Fluoroanilino)-4-(4-pyridylmethyl)phthalazine; 1-(3-Hydroxyanilino)-4-(4-pyridyl methyl)phthalazine; 1-(4-Hydroxyanilino)-4-(4-pyridylmethyl)phthalazine; 1-(3-Aminoanilino)-4-(4-pyridyl methyl)phthalazine; 1-(3,4-Dichloroanilino)-4-(4-pyridylmethyl)phthalazine; 1-(4-Bromoanilino)-4-(4-pyridylmethyl)phthalazine; 1-(3-Chloro-4-methoxyanilino)-4-(4-pyridylmethyl)phthalazine; 1-(4-Cyanoanilino)-4-(4-pyridylmethyl)phthalazine; 1-(4-Methylanilino)-4-(4-pyridylmethyl)phthalazine; and also 1-(3-Chloro-4-fluoroanilino)-4-(4-pyridylmethyl)phthalazine; 1-(3-Methylanilino)-4-(4-pyridylmethyl)phthalazine.

In a further, preferred embodiment, the c-kit inhibitor is compound CT-53518 (MLN-518, Millenium), which is in clinical trials, or a derivative thereof CT-53518 has the following formula

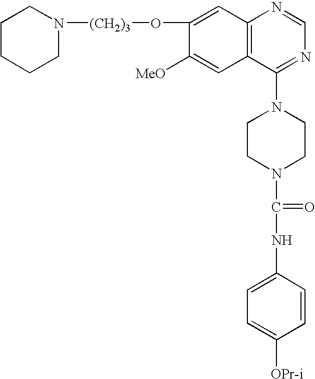

and is known as 1-piperazinecarboxamide, 4-[6-methoxy-7-[3-(1-piperidinyl)propoxy]-4-quinazolinyl]-N-[4-(1-methylethoxy)phenyl]-(9CI); MLN 518; Tandutinib; [4-[6-Methoxy-7-(3-piperidylpropoxy)quinazolin-4-yl]piperazinyl]-N-[4-(methylethoxy)phenyl]carboxamide

Derivatives thereof include nitrogen-containing heterocyclic compounds as described in WO02/016351, having the following formula:

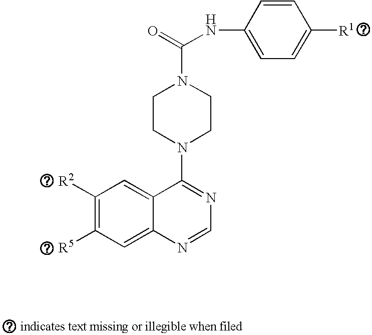

wherein
R¹ is a member selected from the group consisting of:
—CN, —X, —CX . . . 3, —R5, —CO . . . 2R5, —C(O)R5, —So . . . 2R5, —O—C . . . 1-8 alkyl that is straight or
branched chained, —O-phenyl, —O-naphthyl, —O-indolyl and —O-isoquinolinyl;
X is a halogen;
R5 is hydrogen or a Cl—, alkyl that is straight or branched chained;
R2 and R4 are each independently a member selected from the group consisting of:
—O—CH₃, —O—CH₃, —O—CH₂—CH═CH₂, —O—CH₂—C═CH, —O(CH₂)ₙ—SO₂—R⁵, —O—CH₂—CH(R⁶)CH₂—R³ and —O(—CH₂)ₙ—R³;
   R6 is —OH, —X, or a C . . . 1-8 alkyl that is straight or branched chained;
   n is 2 or 3;
   R3 is a member selected from the group consisting of:
   —OH, —O—CH . . . 3, —O—CH . . . 2-CH . . . 3, —NH . . . 2, —N(—CH . . . 3) . . . 2, —NH(—CH . . . 2-phenyl), —NH(-Phenyl), —CN,

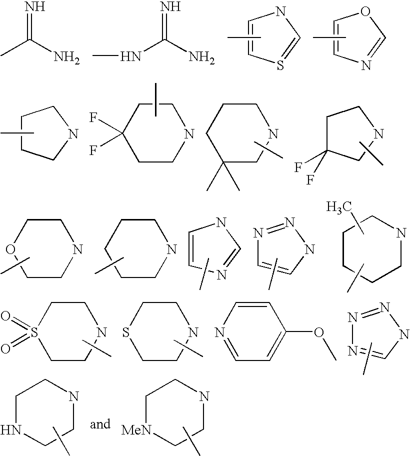

Particular examples of compounds according to the above formula are those in which R1 is a member selected from the group consisting of CN, —O-methyl, —O-ethyl, —O-propyl, —O-isopropyl, —O-butyl, —O-t-butyl, —O-isoamyl, 1-naphthyloxy, 2-naphthyloxy, 4-indolyloxy, 5-indolyloxy, 5-isoquinolyloxy, and position isomers and homologs thereof, and all pharmaceutically acceptable isomers, salts, hydrates, solvates and pro-drug derivatives of such compounds.

Specific examples of such compounds include: N-(4-indol-5-yloxyphenyl){4-[6-methoxy-7-(2-methoxyethoxy)quinazolin-4-yl]piperazinyl}carboxamide;
N-(4-indol-4-yloxyphenyl){4-[6-methoxy-7-(2-methoxyethoxy)quinazoli-n-4-yl]piperazinyl}carboxamide;
{4-[6-methoxy-7-(2-methoxyethoxy)quinazolin-4-yl]piperazinyl}-N-(4-naphthyloxyphenyl)carboxamide;
{4-[6-methoxy-7-(2-methoxyethoxy)quinazolin-4-yl]piperazinyl}-N-(4-(2-naphthyloxy)phenyl)carboxamide;
N-(4-(5-isoquinolyloxy)phenyl){4-[6-methoxy-7-(2-methoxyethoxy)quinazolin-4-yl]piperazinyl}carboxamide;
{4-[6-methoxy-7-(2-methoxyethoxy)quinazolin-4-yl]piperazinyl}-N-(4-phenoxyphenyl)carboxamide;
{4-[6-methoxy-7-(2-methoxyethoxy)quinazolin-4-yl]piperazinyl}-N-[4-(methylethoxy)phenyl]carboxamide;
N-(4-cyanophenyl){4-[6-methoxy-7-(2-methoxyethoxy)quinazolin-4-yl]p-piperazinyl)carboxamide;
{4-[6-methoxy-7-(2-piperidylethoxy)quinazolin-4-yl]piperazinyl}-N-[-4-methylethoxy)phenyl]carboxamide;
N-(4-cyanophenyl){4-[6-methoxy-7-(2-piperidylethoxy)quinazolin-4-yl-]piperazinyl}carboxamide;
{4-[6-methoxy-7-(3-piperidylpropoxy)quinazolin-4-yl]piperazinyl}-N-[4-(methylethoxy)phenyl]carboxamide;
{4-[6-methoxy-7-(3-morpholin-4-yl]propoxy)quinazolin-4-ylpiperazinyl-}-N-[4-(methylethoxy)phenyl]carboxamide;
N-(4-cyanophenyl){4-[6-methoxy-7-(3-morpholin-4-yl]propoxy)quinazolin-4-yl]piperazinyl}carboxamide;
{4-[6-methoxy-7-(3-pyrrolidinylpropoxy)quinazolin-4-yl]piperazinyl)-N-[4-(methylethoxy)phenyl]carboxamide;
N-(4-cyanophenyl){4-[6-methoxy-7-(2-(1,2,3,4-tetraazol-2-yl)ethoxy)-quinazolin-4-yl]piperazinyl}carboxamide;
N-(4-cyanophenyl){4-[6-methoxy-7-(2-(1,2,3,4-tetraazolyl)ethoxy)quinazolinyl]piperazinyl}carboxamide;
{4-[6-methoxy-7-(2-(1,2,3,4-tetraazolyl)ethoxy)quinazolin4-yl]piperazinyl)-N-[4-(methylethoxy)phenyl]carboxamide;
{4-[6-methoxy-7-(2-(1,2,3,4-tetraazol-2-yl)ethoxy)quinazolin4-yl]piperazinyl)-N-[4-(methylethoxy)phenyl]carboxamide;
(4-{7-[3-(4,4-difluoropiperidyl)propoxy]-6-methoxyquinazolin-4-yl}piperazinyl)-N-[4-(methylethoxy)phenyl]carboxamide;
{4-[6-methoxy-7-(3-piperazinylpropoxy)quinazolin-4-yl]piperazinyl)-N-[4-(methylethoxy)phenylcarboxamide;
N-(4-cyanophenyl)(4-{6-methoxy-7-[3-(4-methylpiperazinyl)propoxy]quinazolin-yl}piperazinyl)carboxamide;
N-(4-cyanophenyl)(4-[6-methoxy-7-(3-(1,4thiazaperhydroin-4-yl)propoxy)quinazolin-4-yl]piperazinyl}corboxamide;
4-{7-[3-(1,1-dioxo(1,4-thiazaperhydroin-4-yl))propoxy]-6-methoxyquinazolin-4-yl}piperazinyl)-N-(4-cyanophenyl)carboxamide;
N-(4-cyanophenyl)[4-(7-ethoxy-6-methoxyquinazolin-4-yl)piperazinyl]-carboxamide;
[4-(7-ethoxy-6-methoxyquinazolin-4-yl)piperazinyl]-N-[4-(methylethoxy)phenyl]carboxamide;
[4-(7-ethoxy-6-methoxyquinazolin-4-yl)piperazinyl]-N-(4-naphthyloxy-phenyl)carboxamide;
[4-(7-ethoxy-6-methoxyquinazolin-4-yl)piperazinyl]-N-(4-indol-4-ylo-xyphenyl) carboxamide;
[4-(7-ethoxy-6-methoxyquinazolin-4-yl)piperazinyl]-N-(4-phenoxyphenyl)carboxamide;
N-(4-cyanophenyl)[4-(6-methoxy-7-prop-2-enyloxyquinazolin-4-yl)piperazinyl]carboxamide;
(4-(6-methoxy-7-prop-2-enyloxyquinazolin4-yl)piperazinyl]-N-[4-(methylethoxy)phenyl]carboxamide;
[4-(6-methoxy-7-prop-2-enyloxyquinazolin-4-yl)piperazinyl]-N-(4-naphthyloxyphenyl)carboxamide;
N-(4-indol-4-yloxyphenyl)[4-(6-methoxy-7-prop-2-enyloxyquinazolin-4-yl)piperazinyl]carboxamide;
[4-(6-methoxy-7-prop-2-enyloxyquinazolin-4-yl)piperazinyl]-N-(4-phenoxyphenyl)carboxamide;
N-(4-cyanophenyl) [4-(6-methoxy-7-prop-2-ynyloxyquinazolin-4-yl)piperazinyl]carboxamide;
[4-(6-methoxy-7-prop-2-ynyloxyquinazolin-4-yl)piperazinyl]-N-[4-(methylethoxy)phenyl]carboxamide;
[4-(6-methoxy-7-prop-2-ynyloxyquinazolin-4-yl)piperazinyl]-N-(4-naphthyloxyphenyl)carboxamide;
N-(4-indol-4-yloxyphenyl)[4-(6-methoxy-7-prop-2-ynyloxyquinazolin-4-yl)piperazinyl]carboxamide;
[4-(6-methoxy-7-prop-2-ynyloxyquinazolin-4-yl)piperazinyl]-N-(4-phenoxyphenyl)carboxamide;
(4-{6-methoxy-7-[3-(2-methylpiperidyl)propoxy]quinazolin-4-yl}piperazinyl)-N-[4-(methylethoxy)phenyl]carboxamide;
(4-{6-methoxy-7-[3-(2-methylpiperidyl)propoxy]quinazolin-4-ylpiperazinyl)-N-[4-(methylethoxy)phenyl]carboxamide-N-[4-(methylethoxy)phenyl]carboxamide;
N-(4-cyanophenyl)(4-{6-methoxy-7-[3-(2-methylpiperidyl)propoxy]quinazolin-4-ylpiperazinyl)carboxamide;
N-(4-cyanophenyl)(4-{6-methoxy-7-[3-(4-methylpiperidyl)propoxy]quinazolin-4-ylpiperazinyl)carboxamide;
{4-[7-(2-hydroxy-3-piperidylpropoxy6-methoxyquinazolin-4-yl]piperazinyl)-N-[4-(methylethoxy)phenyl]carboxamide;
{4-[7-(2-fluoox-7-3-pip-eddylpropoyl)-6-methprooxyquinazolin-4-yl]piperazinyl)-N-[4-(methylethoxy)phenyl]carboxamide;
[4-(6-methoxy-7-{3-[(2-methylpropyl)sulfonyl]propoxy}quinazolin-4-yl)piperazinyl]-N-[4-(methylethoxy)phenyl]carboxamide;
(4-f6-methoxy-7-[3-(propylsulfonyl)propoxy]quinazolin-4-yl)piperazinyl)-N-[4-(methylethoxy)phenyl]carboxamide;
methyl 4-({4-[6-methoxy-7-(3-pyrrolidinylpropoxy)quinazolin-4-yl]piperazinyl)carbonylamino)benzoate;
N-(4-acetylphenyl){4-[6-methoxy-7-(3-pyrrolidinylpropoxy)quinazolin-4-yl]piperazinyl}carboxamide;
N-4-bromophenyl){4-[6-methoxy-7-(3-pyrrolidinylpropoxy)quinazolin-4-yl]piperazinyl}carboxamide;
{4-[6-methoxy-7-(3-pyrrolidinylpropoxy)qunazolin-4-yl]piperazinyl}-N-[4-(trifluoromethyl)phenyl]carboxamide;
{4-[6-methoxy-7-(3-pyrrolidinylpropoxy)quinazolin-4-yl]piperazinyl)-N-(4-methylphenyl)carboxamide;
(4-[6-methoxy-7-(3-pyrrolidinylpropoxy)quinazolin-4-yl]piperazinyl)-N-[4-(methylsulfonyl)phenyl]carboxamide;
N-4-fluorophenyl){4-[6-methoxy-7-(3-pyrrolidinylpropoxy)quinazolin-4-yl]piperazinyl}carboxamide;
4-({4-[6-methoxy-7-(3-pyrrolidinylpropoxy)quinazolin-4-yl]piperazinyl}carbonylamino)benzoic acid.

A further group of c-kit inhibitors for use in the present invention includes Semaxinib (SU-5416, Sugen) and derivatives thereof. Semaxinib, 2H-Indol-2-one, 3-[(3,5-dimethyl-1H-pyrrol-2-yl)methylene]-1,3-dihydro-(9CI), is a compound of formula:

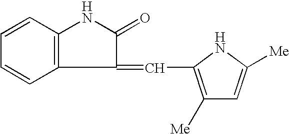

also known as 3-[(3,5-Dimethylpyrrol-2-yl)methylene]indolin-2-one; NSC 696819; Semoxind; Sugen 5416.

Derivatives thereof include protein kinase inhibitors having the following formula, as disclosed in WO03/015608:

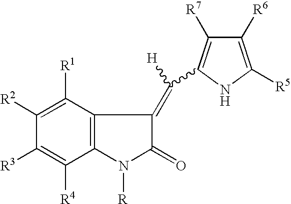

wherein:
R is selected from the group consisting of hydrogen, piperazin-1-ylmethyl, 4-methylpiperazin-1-ylmethyl, piperidin-1-ylmethyl, 2-hydroxymethylpyrrolidin-1-ylmethyl, 2-carboxypyrrolidin-1-ylmethyl, and pyrrolidin-1-ylmethyl;
R1 is selected from the group consisting of hydrogen, halo, alkyl, substituted alkyl cycloalkyl, substituted cyclkoalkyl, aryl, heteroaryl, heteroalicyclic, hydroxy, alkoxy, —C(O)NR8R9, —NR13R14, —(CO)R15, and —(CH2) . . . rR16;
R2 is selected from the group consisting of hydrogen, halo, alkyl, substituted alkyl, trihalomethyl, hydroxy, alkoxy, cyano, —NR13R14, —NR13C(O)R4, —C(O)R15, aryl, heteroaryl, and —S(O) . . . 2NR13R14;
R3 is selected from the group consisting of hydrogen, halogen, alkyl, substituted alkyl, trihalomethyl, hydroxy, alkoxy, aryl, heteroaryl, —NR13R14, —NR13S(O) . . . 2R14, —S(O)2NR13R14, —NR13C(O)R14, —NR13C(O)OR14, —(CO)R15, and —SO . . . 2R19;
R4 is selected from the group consisting of hydrogen, halogen, alkyl, substituted alkyl, hydroxy, alkoxy, and —NR13R14;
R5 is selected from the group consisting of hydrogen, alkyl, substituted alkyl, and —C(O)R10;
R6 is selected from the group consisting of hydrogen, alkyl, substituted alkyl, and —C(O)R10;
R7 is selected from the group consisting of hydrogen, alkyl, substituted alkyl, aryl, heteroaryl, —C(O)R7, and —C(O)R10 provided that when R is hydrogen then at least one of R5, R6 and R7 is —C(O)R10; or
R6 and R7 may combine to form a group selected from the group consisting of —(CH₂)4-, —(CH₂)5- and —(CH₂)6-;
R8 and R9 are independently selected from the group consisting of hydrogen, alkyl, substituted alkyl, and aryl;
R10 is selected from the group consisting of hydroxy, alkoxy, aryloxy, —N(R11)(alkylene)nR12 wherein the alkylene group is optionally substituted with a hydroxy group, and —NR13R14;
R11 is selected from the group consisting of hydrogen, alkyl, and substituted alkyl;
R12 is selected from the group consisting of —NR13R14, hydroxy, —C(O)R15, aryl, heteroaryl, —N+(O—)R13R14, —N(OH)R13, and —NHC(O)R18 (wherein R18 is alkyl, substituted alkyl, haloalkyl, or aralkyl);
R13 and R14 are independently selected from the group consisting of hydrogen, alkyl, substituted alkyl, lower alkyl substituted with hydroxyalkylamino, cyanoalkyl, cycloalkyl, substituted cycloalkyl, aryl and heteroaryl; or
R13 and R14 may combine to form a heterocyclo group;
R15 is selected from the group consisting of hydrogen, hydroxy, alkoxy and aryloxy;
R16 is selected from the group consisting of hydroxy, —NR13R14, —C(O)R15, and —C(O)NR13R14;
R17 is selected from the group consisting of alkyl, substituted alkyl, cycloalkyl, aryl and heteroaryl;
R19 is selected from the group consisting of alkyl, substituted alkyl, aryl, aralkyl, heteoaryl, or heteroaralkyl; and
n and r are independently 1, 2, 3, or 4.

Representative compounds are shown in Table 1 of PCT application WO03/015608. Specific examples of such protein kinase inhibitors include 3-(3,5-dimethylpyrrol-2-ylmethylidene)-2-indolinone (su 5416); 3-[3,5-dimethyl-4-(2-carboxyethyl)pyrrol-2-ylmethylidene]-2-indolinone (su 6668), and 3-[3-(2-carboxyethyl)-5-methylpyrrol-2-ylmethylidene)-2-indolinone.

Additional examples of c-kit inhibitors that can be used in the present invention are disclosed in WO2005/020921, WO2004/046120, WO2005/030776; WO2005/013982; WO2004/058749, WO2005/021531, WO2005/021537, WO2004/063330, for instance. Moreover, further c-kit inhibitors may be selected or identified using conventional screening assays, including the biological assay as described in example 2 of the present application. Furthermore, it should be understood that all position isomers and homologs thereof, as well as all pharmaceutically acceptable isomers, salts, free-bases, hydrates, solvates and pro-drug derivatives of the compounds cited above are also encompassed for use in the present application.

Within the context of this invention, the term “neuroactive” compound designate any compound having biological activity against a neurological disorder, particularly any compound having clinical activity against a neurological disorder. Such compounds may, in particular, directly or indirectly improve nerve function or structure. Such compounds include, without limitation, neuro-protective agents, immunosuppressive drugs, immunomodulatory drugs, corticosteroids, cytokines, as well as, generally, any demyelinating disease modifying treatment, i.e., compounds that modify the course of the disease.

By “corticosteroid” is meant any naturally occurring or synthetic steroid hormone which can be derived from cholesterol and is characterized by a hydrogenated cyclopentanoperhydrophenanthrene ring system. Naturally occurring corticosteriods are generally produced by the adrenal cortex. Synthetic corticosteriods may be halogenated. Corticosteroids may have glucocorticoid and/or mineralocorticoid activity.

Exemplary corticosteroids include, for example, dexamethasone, betamethasone, triamcinolone, triamcinolone acetonide, triamcinolone diacetate, triamcinolone hexacetonide, beclomethasone, dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone 21-hemisuccinate free acid, prednisolone metasulphobenzoate, prednisolone terbutate, and triamcinolone acetonide 21-palmitate.

Preferred examples of corticosteroids are prednisone and IV methylprednisolone.

Examples of immunosuppressive drugs include, without limitation, methotrexate, azathioprine, cyclophosphamide, and cladribine, which are generally used for severe progressive forms of demyelinating diseases.

Other neuroactive agents within the context of this invention include neuroprotective agents such as oral myelin, Copaxone (Glatiramer Acetate from Teva), Tysabri (Biogen/Elan), Novantrone (Serono), Teriflunomide (Aventis), Cladribine (Serono/IVAX), 683699 (T-0047) of GSK/Tanabe Seiyaku, Daclizumab (Roche), Laquinimod (Active Biotech) and ZK-117137 (Schering AG). These compounds are all on the market or in clinical trials to treat MS.

Other neuroactive compounds according to the present invention include immunomodulatory drugs. In this respect, particular neuroactive compounds for use in the present invention include FTY720 (fingolimod) as well as derivatives thereof. FTY720 which is in phase II to treat MS (Novartis) has the following formula:

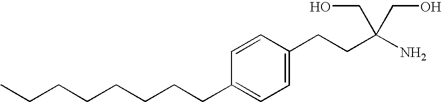

FTY720 (2-amino-2-[2-(4-octylphenyl)ethyl]-1,3-propanediol) has been identified as an orally active immunosuppressant (see, e.g., WO 94/08943; WO 99/36065) obtained by chemical modification of myriocin. Derivatives of FTY720 include 2-amino-1,3-propanediol compounds as described in WO94/08943, having the following formula, as well as any pharmaceutically acceptable salts thereof:

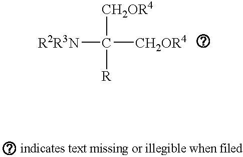

wherein R is an optionally substituted straight- or branched carbon chain which may have, in the chain, a bond, a hetero atom or a group selected from the group consisting of a double bond, a triple bond, oxygen, sulfur, sulfinyl, sulfonyl, —N(R6)- where R6 is hydrogen, alkyl, aralkyl, acyl or alkoxycarbonyl, carbonyl, optionally substituted arylene, optionally substituted cycloalkylene, optionally substituted heteroarylene and an alicycle thereof, and which may be substituted, at the chain end thereof, by a double bond, a triple bond, optionally substituted aryl, optionally substituted cycloalkyl, optionally substituted heteroaryl or an alicycle thereof; an optionally substituted aryl, an optionally substituted cycloalkyl, an optionally substituted heteroaryl or an alicycle thereof; and

R2, R3, R4 and R5 are the same or different and each represents a hydrogen, an alkyl, an aralkyl, an acyl or an alkoxycarbonyl or, R4 and R5 may be bonded to form an alkylene chain which may be substituted by an alkyl, aryl or aralkyl.

The above, optionally substituted straight- or branched carbon chains, may have a substituent selected from the group consisting of alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxyimino, hydroxy, carboxy, optionally substituted aryl, optionally substituted aryloxy, optionally substituted cycloalkyl, optionally substituted heteroaryl and an alicycle thereof; the aforementioned optionally substituted arylene, optionally substituted cycloalkylene, optionally substituted heteroarylene and an alicycle thereof may have a substituent selected from the group consisting of alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxy and carboxy; and the optionally substituted aryl, optionally substituted aryloxy, optionally substituted cycloalkyl, optionally substituted heteroaryl and an alicycle thereof may have a substituent selected from the group consisting of alkyl, alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxy and carboxy.

Specific examples of such 2-amino-1,3-propanediol compounds include 2-amino-2-[2-(4-heptylphenyl)ethyl]-1,3-propanediol, 2-amino-2-[2-(4-octylphenyl)ethyl]-1,3-propanediol, 2-amino-2-[2-(4-nonylphenyl)ethyl]-1,3-propanediol 2-amino-2-[2-(4-decylphenyl)ethyl]-1,3-propanediol, 2-amino-2-[2-(4-undecylphenyl)ethyl]-1,3-propanediol, 2-amino-2-[2-(4-dodecylphenyl)ethyl]-1,3-propanediol, 2-amino-2-[2-(4-tridecylphenyl)-ethyl]-1,3-propanediol, 2-amino-2-[2-(4-tetradecylphenyl)ethyl]-1,3-propanediol, 2-amino-2-[2-(4-hexyloxyphenyl)ethyl]-1,3-propanediol, 2-amino-2-[2-(4-heptyloxyphenyl)ethyl]-1,3-propanediol, 2-amino-2-[2-(4-octyloxyphenyl)ethyl]-1,3-propanediol, 2-amino-2-[2-(4-nonyloxyphenyl)ethyl]-1,3-propanediol, 2-amino-2-[2-(4-decyloxyphenyl)ethyl]-1,3-propanediol, 2-amino-2-[2-(4-undecyloxyphenyl)ethyl]-1,3-propanediol, 2-amino-2-[2-(4-dodecyloxyphenyl)ethyl]-1,3-propanediol, 2-amino-2-[2-(4-tridecyloxyphenyl)ethyl]-1,3-propanediol, 2-amino-2-[2-(4-(8-fluorooctyl)phenyl)ethyl]-1,3-propanediol, 2-amino-2-[2-(4-(12-fluorododecyl)phenyl)ethyl]-1,3-propanediol, 2-amino-2-[2-(4-(7-fluoroheptyloxy)phenyl)ethyl]-1,3-propanediol, 2-amino-2-[2-(4-(11-fluoroundecyloxy)phenyl)ethyl]-1,3-propanediol, 2-amino-2-[2-(4-(7-octenyloxy)phenyl)ethyl]-1,3-propanediol, 2-amino-2-[2-(4-heptylphenyl)ethyl]-1,3-propanediol, 2-amino-2-[2-(4-octylphenyl)ethyl]-1,3-propanediol, 2-amino-2-[2-(4-nonylphenyl)ethyl]-1,3-propanediol, 2-amino-2-[2-(4-decylphenyl)ethyl]-1,3-propanediol, 2-amino-2-[2-(4-undecylphenyl)ethyl]-1,3-propanediol, 2-amino-2-[2-(4-dodecylphenyl)ethyl]-1,3-propanediol, 2-amino-2-[2-(4-heptyloxyphenyl)ethyl]-1,3-propanediol, 2-amino-2-[2-(4-octyloxyphenyl)ethyl]-1,3-propanediol, 2-amino-2-[2-(4-nonyloxyphenyl)ethyl]-1,3-propanediol, 2-amino-2-[2-(4-undecyloxyphenyl)ethyl]-1,3-propanediol, and 2-amino-2-[2-(4-(7-octenyloxy)phenyl)ethyl]-1,3-propanediol, as well as any pharmaceutically acceptable salts thereof.

Other neuroactive compounds according to the present invention include cytokines. The cytokine may be any cytokine, such as interleukin 1 (IL-1), IL-2, IL-3, IL-5, IL-6, IL-7, IL-8, IL-9, IL-12, IL-14, IL-17, granulocyte macrophage colony stimulating factor, monocyte chemoattractant protein-1, interferons, tumor necrosis factors as described in greater details below.

A particular and preferred type of neuroactive compound is interferon. The terms “interferon (IFN)” and “interferon-beta (IFN-beta)”, as used herein, are intended to include fibroblast interferon in particular of human origin, as obtained by isolation from biological fluids or as obtained by DNA recombinant techniques from prokaryotic or eukaryotic host cells, as well as their salts, functional derivatives, variants, analogs and active fragments. A particular type of interferon beta is interferon beta-1a.

IFN-beta suitable in accordance with the present invention is commercially available, e.g., as Rebif® (Serono), Avonex® (Biogen) or Bertaseron/Betaferon® (Schering). The use of interferons of human origin is also preferred in accordance with the present invention. Rebif® (recombinant human interferon-) is the latest development in interferon therapy for multiple sclerosis (MS) and represents a significant advance in treatment. Rebif® is interferon (IFN)-beta 1a, produced from mammalian cell lines. It was established that interferon beta-1a given subcutaneously three times per week is efficacious in the treatment of Relapsing-Remitting Multiple Sclerosis (RRMS). Interferon beta-1a can have a positive effect on the long-term course of MS by reducing number and severity of relapses and reducing the burden of the disease and disease activity as measured by MRI.

Particular examples of neuroactive compounds for use in the present invention therefore include the following FDA approved agents: beta interferons (Betaseron®, Berlex; Avonex®, Biogen; Rebif®, Serono) and Glatimarer Acetate (Copaxone®, Amgen).

In a most preferred embodiment, the neuroactive compound is an interferon, more preferably a human interferon, even more preferably a recombinant human interferon, such as recombinant human interferon beta-1a.

Accordingly, a particular aspect of this invention is a method of treating a neurological disease, particularly a demyelinating disease in a subject in need of such treatment, comprising administering to the subject a therapeutically effective amount of a combination of an interferon (preferably interferon-beta, more preferably interferon-beta 1a) and a c-kit inhibitor.

In another particular aspect of the invention there is provided a method of treating multiple sclerosis in a subject in need of such treatment, comprising administering to the subject a therapeutically effective amount of a combination of an interferon (preferably interferon-beta, more preferably interferon-beta 1a) and a c-kit inhibitor.

In a further particular aspect, the invention relates to a method of treating a neurological disease, particularly a demyelinating disease in a subject in need of such treatment, the method comprising administering to the subject a therapeutically effective amount of a combination of a neuroactive compound and a c-kit inhibitor selected from imatinib; ZK-222584; CT-53518 and Semaxinib.

In a further embodiment, the invention relates to a method of preparing a pharmaceutical treatment for treating a neurological disease, particularly a demyelinating disease in a subject, particularly multiple sclerosis, the method comprising providing a c-kit inhibitor, selected from imatinib; ZK-222584; CT-53518 and Semaxinib and a neuroactive compound selected from an interferon, in a form suitable for administration to a subject.

A further object of this invention relates to an improved method for treating a neurological disease, particularly a demyelinating disease in a subject receiving interferon therapy, the improvement comprising administering to said patient an effective amount of a c-kit inhibitor.

The invention also relates to the use of a therapeutically effective amount of a neuroactive compound for the manufacture of a pharmaceutical composition for treating a neurological disease, particularly a demyelinating disease in a subject in need of such treatment, wherein the subject has a susceptibility alteration in a c-kit gene.

The invention further relates to the use of a c-kit inhibitor for the manufacture of a medicament for treating a demyelinating disease in a mammalian subject, preferably a human subject. Preferably, the demyelinating disease is multiple sclerosis and/or the c-kit inhibitor is selected from the group consisting of imatinib; ZK-222584; CT-53518 and Semaxinib.

According to particular embodiments, the above methods or use further comprise the administration, to the subject, of a corticosteroïd.

The dosage, formulation and administration routes of the active agents used in the present invention may be adjusted by the skilled artisan, based on data available in the art and depending on the subject and disease.

In particular, the active ingredients of the invention can be administered to an individual by intradermal, transdermal (e.g. in slow release formulations), intramuscular, intraperitoneal, intravenous, subcutaneous, oral, epidural, topical, and intranasal routes. Any other therapeutically efficient route of administration can be used, for example absorption through epithelial or endothelial tissues or by gene therapy wherein a DNA molecule encoding the active agent (where such agent is a polypeptide) is administered to the patient (e.g. via a vector), which causes the active agent to be expressed and secreted in vivo. In addition, protein(s) according to the invention can be administered together with other components of biologically active agents such as pharmaceutical acceptable surfactants, excipients, carriers, diluents and vehicles.

The subcutaneous injection is preferred in accordance with the present invention.

The active agents may be formulated or conditioned in any suitable, pharmaceutically acceptable excipient(s) or vehicle(s). In this regard, the term “pharmaceutically acceptable” is meant to encompass any carrier (e.g., support, substance, solvent, etc.) which does not interfere with effectiveness of the biological activity of the active ingredient(s) and that is not toxic to the host to which it is administered. For example, for parenteral administration, the active compounds(s) may be formulated in a unit dosage form for injection in vehicles such as saline, dextrose solution, serum albumin and Ringer's solution.

For parenteral (e.g., intravenous, subcutaneous, intramuscular) administration, the active agent(s) can be formulated as a solution, suspension, emulsion or lyophilised powder in association with a pharmaceutical acceptable parenteral vehicle (e.g., water, saline, dextrose solution) and additives that maintain isotonicity (e.g., mannitol) or chemical stability (e.g., preservatives and buffers). The formulation is sterilized by commonly used techniques.

The bioavailability of the active agent(s) according to the invention can also be ameliorated by using conjugation procedures which increase the half-life of the molecule in the human body, for example linking the molecule to polyethyleneglycol, as described in the PCT Patent Application WO92/13095.

The dosage administered, as single or multiple doses, to an individual will vary depending upon a variety of factors, including pharmacokinetic properties, the route of administration, patient conditions and characteristics (sex, age, body weight, health, size), extent of symptoms, concurrent treatments, frequency of treatment and the effect desired.

Standard dosages of human IFN-beta presently used in the treatment of relapsing-remitting MS are ranging from 80 000 IU/kg and 200 000 IU/kg per day or 6 MIU (million international units) and 12 MIU per person per day or 22 to 44 μg per person. In accordance with the present invention, IFN may be administered on the basis of a dosage of about 1 to 50 μg, preferably of about 10 to 50 μg, more preferably of about 10 to 45 μg per person, three times per week. The preferred route of administration is subcutaneous administration, administered three times a week. A further preferred route of administration is the intramuscular administration, which may be applied once a week.

In accordance with the present invention, where IFN is recombinant IFN-β1b produced in E. coli, commercially available under the trademark Betaseron, it may preferably be administered sub-cutaneously every second day at a dosage of about of 50 to 500 μg, more preferably 250 to 300 μg (or 8 MIU to 9.6 MIU) per person.

In accordance with the present invention, where IFN is recombinant IFN-β1a, produced in Chinese Hamster Ovary cells (CHO cells), commercially available under the trademark Avonex, it may preferably be administered intramuscularly once a week at a dosage of about of 5 to 50 μg, more preferably of about 30 μg to 33 μg (or 6 MIU to 6.6 MIU) per person.

In accordance with the present invention, when IFN is recombinant IFN-β1a, produced in Chinese Hamster Ovary cells (CHO cells), commercially available under the trademark Rebif, it may preferably be administered sub-cutaneously three times a week (TIW) at a dosage of 10 to 100 μg, preferably of about 22 to 44 μg (or 6 MIU to 12 MIU) per person.

Another possibility of carrying out the present invention is to activate endogenously the genes for the compounds of the invention, e.g., IFN. In this case, a vector or compound for inducing and/or enhancing the endogenous production of IFN in a cell is used for treatment of a demyelinating disease.

With regard to corticosteroids, oral prednisone may be administered at 60 to 100 mg/day tapered over 2 to 3 weeks or IV methylprednisolone may be administered at 500 to 1000 mg/day for 3 to 5 days, for instance.

The substances of the invention may be administered daily or every other day, of less frequently. Preferably, one or more of the substances of the invention are administered one, twice or three times per week.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, the substances of the invention can be administered prophylactically or therapeutically to an individual prior to, simultaneously or sequentially with other therapeutic regimens or agents (e.g. multiple drug regimens), in a therapeutically effective amount. In a particular embodiment, the neuroactive compound is administered prior to the c-kit inhibitor. When simultaneous administration is performed, the active agents can be administered in the same or different compositions.

As will be disclosed, the invention may be used in any mammalian subject, including human subjects, and provide improved therapeutic approach to the treatment of neurological diseases. The administration of a pharmaceutical combination of the invention results not only in a beneficial effect, e.g., a synergistic therapeutic effect, e.g., with regard to alleviating, delaying progression of or inhibiting the symptoms, but also in further surprising beneficial effects, e.g., fewer side-effects an improved quality of life or a decreased morbidity, compared with a monotherapy applying only one of the pharmaceutical active ingredients used in the combination of the invention. A further benefit is that lower doses of the active ingredients of the combination of the invention can be used, for example, that the dosages need not only often be smaller but are also applied less frequently, which may diminish the incidence or severity of side-effects. This is in accordance with the desires and requirements of the patients to be treated.

A further aspect of this invention is a method of detecting the presence of or predisposition to a neurological disease, particularly a demyelinating disease in a subject, the method comprising detecting in vitro or ex vivo the presence or a susceptibility alteration in a c-kit gene or polypeptide in a sample from the subject, the presence of such an alteration being indicative of the presence of or predisposition to a neurological disease, particularly a demyelinating disease in the subject.

The invention also relates to a method of assessing the response or responsiveness of a subject to a treatment of a neurological disease, particularly a demyelinating disease, the method comprising detecting in vitro or ex vivo the presence or a susceptibility alteration in a c-kit gene or polypeptide in a sample from the subject, the presence of such an alteration being indicative of a responder subject.

The susceptibility alteration in a c-kit gene or polypeptide may be any susceptibility marker in said gene or polypeptide, i.e., any nucleotide or amino acid alteration associated to a neurological disease, particularly a demyelinating disease. An alteration in the c-kit gene may be any form of mutation(s), deletion(s), rearrangement(s) and/or insertion(s) in the coding and/or non-coding region of the gene, either isolated or in various combination(s). Mutations more specifically include point mutations. Deletions may encompass any region of two or more residues in a coding or non-coding portion of the gene. Typical deletions affect small regions, such as domains (introns) or repeated sequences or fragments of less than about 50 consecutive base pairs, although larger deletions may occur as well. Insertions may encompass the addition of one or several residues in a coding or non-coding portion of the gene. Insertions may typically comprise an addition of between 1 and 50 base pairs in the gene. Rearrangements include for instance sequence inversions. An alteration in the c-kit gene may also be an aberrant modification of the polynucleotide sequence, such as of the methylation pattern of the genomic DNA, allelic loss of the gene or allelic gain of the gene. The alteration may be silent (i.e., create no modification in the amino acid sequence of the protein), or may result, for instance, in amino acid substitutions, frameshift mutations, stop codons, RNA splicing, e.g. the presence of a non-wild type splicing pattern of a messenger RNA transcript, or RNA or protein instability or a non-wild type level of the c-kit polypeptide. Also, the alteration may result in the production of a polypeptide with altered function or stability, or cause a reduction or increase in protein expression levels.

Typical alterations are single nucleotide polymorphisms. In this regard, the present invention now discloses several markers or mutations in the c-kit gene, which are associated with multiple sclerosis. These mutations are reported in tables 2 and 3.

The susceptibility alteration may be detected by a number of techniques which are known per se in the art, including sequencing, selective hybridisation and/or amplification.

Sequencing can be carried out using techniques well known in the art, using automatic sequencers. The sequencing may be performed on the complete gene or, more preferably, on specific domains thereof, typically those known or suspected to carry deleterious mutations or other alterations.

Amplification may be performed according to various techniques known in the art, such as by polymerase chain reaction (PCR), ligase chain reaction (LCR) and strand displacement amplification (SDA). These techniques can be performed using commercially available reagents and protocols. A preferred technique is allele-specific PCR.

The detection methods can be performed in vitro, ex vivo or in vivo, preferably in vitro or ex vivo. They are typically performed on a sample from the subject, such as any biological sample containing nucleic acids or polypeptides. Examples of such samples include fluids, tissues, cell samples, organs, biopsies, etc. Most preferred samples are blood, plasma, saliva, urine, seminal fluid, etc. The sample may be collected according to conventional techniques and used directly for diagnosis or stored. In particular, they may be obtained by non-invasive methods, such as from tissue collections. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

The sample is typically contacted with probes or primers as disclosed above. Such contacting may be performed in any suitable device, such as a plate, tube, well, glass, etc. The contacting may performed on a substrate coated with said specific reagents, such as a nucleic acid array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids of the sample.

The finding of an altered c-kit gene or RNA or polypeptide in the sample is indicative of the presence, predisposition or stage of progression of a neurological disease, particularly a demyelinating disorder in the subject, or defines a responsive group. Typically, one only of the above-disclosed markers is assessed, or several of them, in combination(s).

The invention also encompasses kits for the identification of a genetic polymorphism pattern at the c-kit gene associated with increased risk of the presence of or predisposition to a neurological disease, particularly a demyelinating disease in a subject, said kits comprising:

(a) DNA sample collecting means, and
(b) means for determining a genetic polymorphism pattern for the c-kit gene.

Further aspects and advantages of the invention will be disclosed in the following examples, which should be regarded as illustrative and not limiting the scope of this application. All publications or patent applications cited in the present application are hereby specifically incorporated therein by reference.

### EXAMPLES

### Example 1

### Materials and Methods

### 1.1 Collections of Patients and DNA Banking—Subjects

The study comprised three collections of unrelated patients with multiple sclerosis (MS) and unrelated healthy controls recruited from the neurological Department of Rennes (France: 314 cases; 353 controls), Huddinge (Sweden: 279 cases; 301 controls) hospitals and SeraCare (USA: 289 cases; 289 controls). Table 1 provides a summary for the description and stratification study of the different collections.

Informed consent was given by each individual participating in the study, according to the Helsinki Convention (1964).

The following variables were recorded for each patient: sex, ethnic background, family history with regards to MS, diagnostic category, disease course, age at disease onset, results of cerebrospinal fluid (CSF) and Magnetic Resonance Imaging (MRI) examination, Expanded Disability Status scale (EDSS) score and disease duration at last inter-relapse clinical examination.

Disease courses were classified as relapsing-remitting (RR), relapsing-secondary progressive (SP), or primary-progressive (PP) as follows:
RR: relapses with full recovery or with a residual deficit and lack of progression between relapses;
SP: initial RR MS followed by progression;
PP: Disease progression from onset

Selected for this study were patients with Primary progressive type, remitting-relapsing type or secondary progressive type MS, who have been diagnosed as MS according to the criteria of Mc Donald et al. (2001).

Each subject was assessed clinically by the Kurtzke Expanded Disability Status Scale (EDSS) (Kurtzke, 1983), using the latest data available.

### Rennes Collection:

Each patient and control subject included in the analysis had to be born in Bretagne, France as well as their parents and grand-parents.

The female/male ratio in the patient group was 2.14 (214 Females & 100 Males) with a mean age of 44 [19; 68] years and in the control group 2.07 (241 Females & 116 Males) with a mean age of 35 [18; 56] years.

### Huddinge Collection:

All participants in this study were drawn from a homogeneous population of Huddinge, Sweden.

The female/male ratio in the patient group was 2.4 (196 Females & 83 Males) with a mean age of 47 [22; 75].

The control group in Huddinge collection included 301 (214 Females & 87 Males) healthy volunteers and the Female/male ratio was 2.5. Ages ranged from 22 to 73 years with a mean age of 47 years.

### Seracare Collection:

All the subjects included in the study were Caucasian from USA.

The group of cases included 289 subjects with a sex ratio of 5.7 (246 females and 43 males) and a mean age of 50 [32; 74] years.

The group of healthy volunteers included 289 individuals with a sex ratio of 5.7 (246 females and 43 males) and a mean age of 48.7 [36; 75] years.

TABLE 1
Description and stratification study of the different collections

| | | | | | | | Genetic |
|---|---|---|---|---|---|---|---|
| | | | Clinical | | | Age at | Homogeneity |
| Sample | Status | Sex | Form* | | Mean age | onset | Study |
| Rennes | 314 cases | 214 females | 33 | PP | 49 [22; 70] | 39 [18; 57] | FST = 0.00036 |
| N = 667 | (sex ratio | (68%) | 72 | RP | 50 [20; 72] | 31 [15; 46] | pvalue: 7.33E−02 |
| | 2.2) | | 109 | RR | 39 [18; 81] | 29 [10; 66] | (NS) |
| | | 100 males | 31 | PP | 49 [22; 70] | 37 [14; 54] | Test Pritchard- |
| | | | 33 | RP | 50 [20; 72] | 30 [16; 47] | Rosenberg |
| | | | 36 | RR | 39 [18; 81] | 28 [18; 54] | pvalue: 1.27E−01 |
| | 353 | 238 females | — | | 35 [18; 50] | — | (NS) |
| | controls | (67%) | | | | | 20 000 permutations |
| | (sex ratio | 115 males | — | | 36 [18; 56] | — | 85 random alterations |
| | 2.1) | | | | | | |
| Huddinge | 279 cases | 196 females | 9 | PP | 59 [45; 71] | — | FST = −0.000044 |
| N = 580 | (sex ratio | (70%) | 54 | RP | 54 [33; 73] | — | pvalue: 5.40E−01 |
| | 2.4) | | 133 | RR | 42 [22; 73] | — | (NS) |
| | | 83 males | 11 | PP | 59 [48; 74] | — | Test Pritchard- |
| | | | 25 | RP | 52 [35; 75] | — | Rosenberg |
| | | | 47 | RR | 44 [23; 66] | — | pvalue: 5.00E−01 |
| | 301 | 214 females | — | | 46 [22; 72] | — | (NS) |
| | controls | (71%) | | | | | 20 000 permutations |
| | (sex ratio | 87 males | — | | 48 [23; 73] | — | 83 random alterations |
| | 2.5) | | | | | | |
| SeraCare | 289 cases | 246 females | 14 | PP | 52 [36; 70] | 39 [22; 58] | FST = 0.00014 |
| N = 578 | (sex ratio | (85%) | 34 | RP (or SP) | 52 [36; 66] | 39 [23; 51] | pvalue: 6.81E−01 |
| | 5.7) | | 197 | RR | 48 [31; 74] | 39 [10; 64] | (NS) |
| | | | 1 | benign | 49 | 32 | Test Pritchard- |
| | | 43 males | 2 | PP | 61 | [28; 57] | Rosenberg |
| | | | 9 | RP | 52 [37; 61] | 42 [22; 55] | pvalue: 7.00E−01 |
| | | | 30 | RR | 42 [32; 71] | 39 [15; 62] | (NS) |
| | | | 2 | benign | [60; 64] | [43; 52] | 20 000 permutations |

| | | | | | |
|---|---|---|---|---|---|
| 289 | 246 females | — | 48 [36; 75] | — | 80 random alterations |
| controls | (85%) | | | | |
| (sex ratio | 43 males | — | 50 [36; 70] | — | |
| 5.7) | | | | | |

NS, Non significative P value
*Clinical forms:
PP: Primary progressive
RP: Relapsing Progressing (or SP: Secondary Progressive)
RR: Relapsing Remitting

All the Fst values found for each collection indicate that these samples are genetically homogeneous. They can be therefore used in association analysis.

### 1.2. DNA Extraction:

Genomic DNA was extracted from EDTA anticoagulated peripheral blood according to a standard proteinase K digestion and a modified salting out extraction method of Miller and co-workers (1988).

### 2.1 Genotyping

### 2.1.1 Methods for Stratification Analyses: Beckman UHT Protocol:

### Assay Design

Design of the two PCR primers and one SNP-IT primer for each marker set was performed using Autoprimer.com (http://www.autoprimer.com). The Autoprimer.com design engine reads each sequence and designs three primers; forward and reverse PCR primers and a SNP-IT primer for the single base extension step. Once primers are picked for each sequence, they are then assembled into groups of 12 by SNP extension type (e.g., A/G, T/C).

Each group, or panel of 12 markers, must be of the same extension type for processing on the UHT since each extension mix contains two labeled terminators (Bodipy-Fluorescein and TAMRA). Each group of twelve is referred to as a panel of markers. Autoprimer.com automatically optimizes the grouping of the markers by extension mix and appends tag sequences to the 5′ ends of the SNP-IT primers, which are complementary to the tags immobilized on the microarray plate.

### PCR

A five-microliter PCR was performed in 384-well plates (MJ Research, Watertown, Mass., USA) using 75-uM dNTPs and 0.5 U AmpliTaq® Gold (Applied Biosystems) in 1×PCR buffer. Two nanograms of genomic DNA were used in each reaction.

The 24 PCR primers were pooled and added such that each was at a final concentration of 50 nM. Thermal cycling was performed in DNA Engine Tetrad thermal cyclers (MJ Research) using the following program: 95° C. for 5 seconds followed by 45 cycles of 95° C. for 30 seconds; 50°-55° C. for 55 seconds; 72° C. for 30 seconds. The first six cycles used an annealing temperature of 50° C. after which the annealing temperature was increased by 0.2° C. in the subsequent cycles until the annealing temperature reached 55° C. After the last cycle, the reaction was held at 72° C. for 7 minutes followed by a 4° C. hold.

### PCR Clean-Up

Following PCR, 384-well plates were centrifuged briefly to collect the contents and 3 uL of a cocktail containing 0.67 U exonuclease I (USB, Cleveland, Ohio, USA) and 0.33 U shrimp alkaline phosphatase (SAP; USB) was added. Sealed plates were incubated for 30 minutes at 37° C. to degrade residual PCR primers and dNTPs, and 10 minutes at 100° C. to inactivate the enzymes.

### SNP-IT Reaction

To the ExoI/SAP-treated PCR, we added 7 μL of a cocktail containing one TAMRA-labeled and one Bodipy-Fluorescein-labeled nucleotide terminator (PE-NEN, Boston, Mass., and Molecular Probes, Eugene, Oreg., USA), the two remaining unlabeled terminators, 26.6 mM MgCl2, 266 mM Tris-HCl pH 9.5, two allele-specific self-extension control primers, and a thermostable, 3′ exonuclease-deficient polymerase such as Thermo Sequenase (Amersham Biosciences, Piscataway, N.J., USA). The total reaction volume was 15 uL. Plates were re-sealed and thermal cycled using the following program: 96° C. for 3 minutes followed by 45 cycles of 94° C. for 20 seconds; 40° C. for 11 seconds. After the last cycle, the reaction was held at 4° C.

### Hybridization and Washing

Following SNP-IT extension, 8 μL of hybridization buffer (5M NaCl, 0.5 M EDTA, 580 mM morpholinoethane sulphonic acid (MES) pH 6.6, 1×Denhardt's Solution) was added and a portion of the mixture was applied to the well of a UHT microarray plate.

Plates were incubated in a humidified container at 42° C. for 2 hours to promote hybridization of the SNP-IT primers to their complementary immobilized tags. Plates were rinsed with UIHT wash buffer using a conventional plate washer to remove unhybridized material and were then ready for imaging.

### SNPstream UHT Array Imager

The SNPstream Array Imager is based upon a two-laser, two-color approach. Each sample is illuminated with a 488-nm laser beam and subsequently with a 532-nm laser beam to excite the fluorescent oligonucleotides captured on the UIHT microarray plates. The system contains two emission band filters. Fluorescence emission from 488-nm excitation (Bodipy-Fluorescein) is captured in a band 50 nm wide, centered at 535 nm. Fluorescence emission from 532-nm excitation (TAMRA) is captured in a band 55 nm wide, centered at 590 nm. A colorcorrected custom lens, of high numerical aperture and 100-um A 2×3 well area is imaged per frame. Sixty-four 2×3 well images/color are taken per plate for a total of 384 wells. Total time required for the process is approximately seven minutes/plate.

### Data Analysis

Generation of genotype calls from spots detected using the SNPstream UIHT Array Imager involves two discrete steps. First, the location and intensity of a spot within the well and plate is determined for each wavelength; second, a genotype call is made based on the relative fluorescent intensities of each spot. Once a genotype call has been made, results are written to an Oracle® database where the data can easily be retrieved for viewing.

Spot detection is an automatic process performed by UHTImage software. Positive controls in each well are used to align the grids around the 4×4 element array. Once a grid is drawn, each spot is analyzed for morphology (i.e., circular shape and regular pixel intensity across each spot). Spots with low intensity or unusual morphology are marked as empty or fail. For each spot that passes the morphology test, an intensity value is generated and loaded into the UHT database. Failed spots are carried through the analysis but are flagged for the user to review.

Genotype calling is performed once all spot intensities are in the database for each sample within a plate. Each SNP marker is analyzed separately using UHT GetGenossoftware. This software automatically creates genotype calls based on the intensity value of each spot at each wavelength for a given sample. These calls are based on how the sample points cluster when plotted on a X, Y graph where X corresponds to the intensity in the 488-nm channel and Y to that of the 532-nm channel. If a point falls between clusters or the intensity of the point is too low, the sample is failed. Otherwise the point is called as XX, XY, or YY with the X's and Y's being replaced by the actual allele calls (A,C,G,T). UHT GetGenos uses a proprietary algorithm to determine the clusters and the genotypes for each sample. After the genotype calling, the results are stored in the database by microarray plate number, well, and spot location.

### 2.1.2 Methods for Whole Genome Analysis: Affymetrix Method:

### DNA Preparation:

For each individual assayed, 250 ng of genomic DNA are digested separately with 10 U of XbaI or HindIII (New England BioLabs) in volumes of 20 μL for 2 hours at 37° C. Following heat inactivation at 70° C. for 20 minutes, 0.25 μM of XbaI adaptor (5′-ATT ATG AGC ACG ACA GAC GCC TGA TCT-3′ and 5′phosphate-CTA GAG ATC AGG CGT CTG TCG TGC TCA TAA-3′) (Affymetrix), or HindIII adaptor (5′-ATT ATG AGC ACG ACA GAC GCC TGA TCT-3′ and 5′phosphate-AGC TAG ATC AGG CGT CTG TCG TGC TCA TAA-3′) (Affymetrix) are ligated to the digested DNAs with T4 DNA Ligase (New England BioLabs) in 25 μL for 2 hours at 16° C. The ligations are stopped by heating to 70° C. for 20 minutes, and then diluted 4-fold with water. For each ligation reaction, two to three PCRs are run in order to generate >40 μg of PCR products. Each PCR contains 10 μL of the diluted ligation reactions (25 ng of starting DNA) in 100 μL volumes containing 1.0 μM of primer (5′-ATT ATG AGC ACG ACA GAC GCC TGA TCT-3′), 0.30 mM dNTPs, 1.0 mM MgSO₄, 5 U Platinum® Pfx Polymerase (Invitrogen), PCR Enhancer (Invitrogen) and Pfx Amplification Buffer (Invitrogen). 30 cycles of PCRs are run with the following cycling program: 94° C. denaturation for 15 seconds, 60° C. annealing for 30 seconds, and 68° C. extension for 60 seconds. As a check, 3 μL of PCR products are visualized on 2% TBE agarose gels to confirm the size range of amplicons. The PCR products are purified over MinElute 96 UF PCR Purification plates (Qiagen), and recovered in 40 μL of EB buffer (Qiagen). PCR yields are measured by absorbance readings at 260 nm, and adjusted to a concentration of 40 μg per 45 μl. To allow efficient hybridization to 25-mer oligonucleotide probes, the PCR products are fragmented to <100 bp with DNAse I. 0.20 U of DNAse I (Affymetrix) is added to 40 ug of purified PCR amplicons in a 55 μL volume containing Fragmentation Buffer (Affymetrix) for 35 minutes at 37° C., followed by heat inactivation at 95° C. for 15 minutes. Fragmentation products are visualized on 4% TBE agarose gels. The 3′ ends of the fragmented amplicons are biotinlyated by adding 214 μM of a proprietary DNA labeling reagent (Affymetrix) using Terminal Deoxynucleotidyl Transferase (Affymetrix) in 70 μL volumes for 2 hours at 37° C., followed by heat inactivation at 95° C. for 15 minutes.

### Allele Specific Hybridization to Oligonucleotide Arrays

The fragmented and biotinylated PCR amplicons are combined with 11.5 μg/mL human Cot-1 (Invitrogen) and 115 μg/mL herring sperm (Promega) DNAs. The DNAs are added to a hybridization solution containing 2.69 M tetramethylamonium chloride (TMACl), 5.77 mM EDTA, 56 mM MES, 5% DMSO, 2.5×Denhardt's solution, and 0.0115% Tween-20 in a final volume of 260 μL. The hybridization solution was heated to 95° C. for 10 minutes then placed on ice. After warming to 48° C. for 2 minutes, 200 μL of the hybridization solution is injected into cartridges housing the oligonucleotide arrays (Affymetrix GeneChip® 100K Mapping Set: 50 K Array Xba 240 and 50K Array Genotyping over 100,000 SNPs Hind 240). Hybridizations are carried out at 48° C. for 16 to 18 hours in a rotisserie rotating at 60 rpm. Following the overnight hybridization, the arrays are washed with 6×SSPE and 0.01% Tween-20 at 25° C., then more stringently washed with 0.6×SSPE and 0.01% Tween-20 at 45° C. Hybridization signals are generated in a three step signal amplification process: 10 μg/mL streptavidin R-phycoerythrin (SAPE) conjugate (Molecular Probes) is added to the biotinylated targets hybridized to the oligonucleotide probes, and washed with 6×SSPE and 0.01% Tween-20 at 25° C.; followed by the addition of 5 μg/mL biotinylated goat anti-streptavidin (Vector) to increase the effective number of biotin molecules on the target; and finally SAPE is added once again and washed extensively with 6×SSPE and 0.01% Tween-20 at 30° C. The SAPE and antibody were added to arrays in 6×SSPE, 1×Denhardt's solution and 0.01% Tween-20 at 25° C. for 10 minutes each. Following the final wash, the arrays are kept in Holding buffer (100 mM MES, 1M [Na+], 0.01% Tween-20). The washing and staining procedures are run on Affymetrix fluidics stations. Arrays are scanned using GCS3000 scanners with AutoLoaders (Affymetrix). Scan images are processed to get hybridization signal intensity values using GCOS 2.0 software (Affymetrix). The DM genotype calling algorithm is implemented in GenoTyping Tools (GTT) (Affymetrix) and GDAS 3.0 (Affymetrix) analysis software.

### 2.2 Statistical Analysis

Design: We have decided to analyze 2 different populations in parallel to minimize the risk of type I errors (false positives) due to the relatively limited sample size. In our case, the 2 populations have the same euristic value and neither one represents an exploratory or a confirmation sample. Rather they represent 2 complementary views of the same analytical problem and only positive results that are cross-confirmed are retained as valid. The following paragraphs detail the statistics that we applied to perform our analyses. A third population (SeraCare) was studied to further confirm the results.

### Part A: Descriptive Statistics

### 2.2.1 Genetic Homogeneity: FST Test and Pritchard and Rosenberg Test

A stratification effect is a non-homogeneous representation of populations between the case and the control groups due to genetic heterogeneity, which may lead to spurious association results and replication problems.

If cases and controls contain an admixture of different groups (for example, based on ethnicity), we expect to find a consistent pattern of allele-frequency differences between cases and controls, at many random loci throughout the genome, this difference exceeding the significant p-value for association at more than 5% of these random loci.

The power to detect stratification will depend on the number of loci used to test for homogeneity. Consequently, we have chosen a large number of unlinked SNP markers (n=86). These SNPs have been selected under the following conditions:
1 Minor allele frequency >30% (highly polymorph)
2 Inter SNP distance >10 Mb (genetically independent)
3 Location in each chromosome (genome wide scale) but not in a known associated region for the studied disease (not associated with the disease)

All cases and controls were genotyped for all the unlinked genetic markers set using the Beckman technology.

Two methods testing for genetic heterogeneity have been implemented in Serono

Genetics Institute:
1. Fst test (Wright 1951) is an ANOVA-based method. The Fst value quantifies the loss of heterozygosity due to existence of a hierarchical structure. If it is different from 0, it means that the population under study are genetically heterogeneous, since allelic frequencies are different between populations.
2. Pritchard & Rosenberg test (Am. J. Hum. Genet. 65:220-228, 1999) calculates an overall chi-square statistic of allelic frequency differences between cases and controls.

If the Fst and the Pritchard & Rosenberg tests do not show statistically significant results (p-value >5%), cases and controls are considered homogenous and can be used for case-control association study.

However, statistically significant results at these tests do not necessarily mean that these populations must be discarded. Further analyses can assign each subject to a specific subpopulation and identify outliers (Structure software, Pritchard, 2002), that can be removed in order to restore homogeneity.

When the admixture is such that we can not identify clear subpopulations, we can adopt another approach, termed Genomic Control (Devlin and Roeder 1999): given that in the presence of population substructure, the standard chi-square statistic is inflated by a multiplicative factor, which is proportional to the degree of stratification, we can estimate and incorporate this multiplicative factor (lambda) into the disease—marker association tests (by rescaling the chi-square statistic) to correct for background population differences.

### Part B: Inferential Statistics

### 2.2.2 Univariate Analysis

### I. Hardy-Weinberg Equilibrium/Disequilibrium [HWE/D]: Significance in Cases and in Controls

The Hardy-Weinberg law regulating equilibrium (HWE) is the central theory of population genetics, explaining why populations have a stable genetic pattern across generations and is based on four assumptions:
1 Populations are panmict (couples are formed at random) and their gametes meet randomly;
2 Populations are “Infinite” (large population size to minimize sampling variations);
3 There are no selection, mutation, migration (=no allele loss or allele gain);
4 Generations are discrete (no mating between different generations).

According to these hypotheses, the control population used in case-control association studies must respect this equilibrium, if sampled randomly. On the contrary, the population of cases can present some disequilibrium that may point to “mutations” underlying the disease, since cases are not a random representation of the general population.

Accordingly, we tested HWE for each SNP in the control population, and we removed from the study each SNP presenting a deviation from the equilibrium. In fact, any such deviation might be due to several different reasons, but especially to technical issues (e.g. neighbouring SNPs causing imbalance of the polymerase chain reaction products or affecting the genotyping assay). HWE test therefore serves two objectives: data review and quality check as well as detection of possible mutation.

The test described by Weir in Genetic Data Analysis II (Sinauer, 1996) has been implemented using a chi-square statistics (1 df). The SNPs with results showing significant deviation from HWE (pvalue <0.02) were considered in disequilibrium and were not validated, a positive deviation demonstrating an excess of homozygotes (or lack of heterozygotes) and a negative deviation being due to an excess of heterozygotes (or lack of homozygotes).

Hardy-Weinberg equilibrium statistics were calculated separately for cases and controls data and Observed and Expected genotype frequencies were compared using a Pearson's χ² test. A departure from Hardy-Weinberg equilibrium (HWE) in case population may indicate that a mutation had occurred, which could be responsible for increasing the risk for the disease.

### II. Tests on Allelic Frequencies, Genotypic Frequencies, HWD

In the univariate analysis (or Single Point Analysis), SNPs were analysed one by one. The Pearson's 2×2 χ2 test was used to compare allele frequencies between cases and controls, while we used a 3×2 χ2 test for the overall difference in genotype frequencies. The Exact Fisher test was performed wherever the minor expected frequency for each cell of the χ² table is <5.

Additional statistics include (i) the difference between allelic frequencies in cases and in controls (the larger the difference in allelic frequency for a given SNP, the more probable is an association between the genomic region containing that SNP and the disorder), (ii) the Odds Ratio (OR) of the association and (iii) the population Attributable Risk (pAR). The “chosen” allele is the allele for which the frequency is increased in cases compared to controls. Preferred single nucleotide polymorphisms indicative of multiple sclerosis are the chosen alleles of Tables 2 and 3.

We considered a p-value= or <0.05 as threshold to consider the tests as significant for screening, with the only exception relative to HW test where the threshold is = or <0.02.

### III. Mantel Haenszel Test: Comparison of the Significant Findings Across the 2 Populations.

The relationships between genetic susceptibility to MS and allele frequencies have been studied for many markers (N=95 938) in at least one of the two populations (Rennes & Huddinge). Data from most of these SNPs (N=82 925) are available for the two populations (Rennes & Huddinge): therefore, they represent the basis to evaluate associations that are observed in the two populations simultaneously.

We used the Mantel-Haenszel χ² test which was designed for case-control studies in which the effect of an exposure-factor (Allele) on the outcome (MS) is investigated according to a stratification factor (Population).

A program was written at SGI to perform the Mantel-Haenszel test using data from n independent populations (Principles of Biostatistics, Second Edition, Marcello Pagano & Kimberlee Gauvreau, Duxbury-Thomson Learning).

### 2.2.3. Odds Ratio (OR)

By estimating the allelic Odds Ratio (OR) we evaluate the probability of having the disease when carrying a given allele (=chosen [or ‘risk’ ] allele) compared to not carrying it.

An OR higher than 1 shows that the probability of having multiple sclerosis is higher when carrying the ‘risk’ allele [or genotype or haplotype] than when carrying the other ones.

The genotypic OR allows the identification of the ‘risk’ genotype(s) for an associated biallelic marker. The genotypic odds ratio was calculated and Table 2 and 3 below show the marker location and corresponding significant results.

TABLE 2

| | | | MH_pvalue MS | MH_pvalue MS |
|---|---|---|---|---|
| | | | Huddinge + | Huddinge + |
| sitename | chr | DOS | Renne | Renne + SeraCare |
| SNP_A-1655751 | 4 | 55374549 | 0.024 | 0.01 |
| SNP_A-1712954 | 4 | 55451346 | 0.018 | 0.21 |
| SNP_A-1753080 | 4 | 55351790 | 0.00851 | 0.0026 |
| SNP_A-1753252 | 4 | 55352010 | 0.0298 | 0.038 |
| SNP_A-1754613 | 4 | 55352994 | 0.00477 | 0.0012 |

Legend for tables 2 and 3:

| | |
|---|---|
| sitename | affymetrix SNP ID |
| chr | chromosome |
| pos | position in base pairs |
| ChosenAllele | allele frequency increased within cases as |
| | compared to controls |
| allel_freq_diff | allele frequency difference between cases and |
| | controls |
| Allel_test_ExacTest | Ficher's exact test allelic P value |
| OR | odds ratio |
| Gen_test-ExacTest | Ficher's exact test genotypic P value |
| HWE_cases | Hardy-Weinberg P value within cases |
| MH_pvalue | Mantel-Haenszel test P value |

TABLE 3

| KIT | | | | | | | | |
|---|---|---|---|---|---|---|---|---|
| Sitename | chr | pos | ChosenAllele | allel_freq_diff | Allel_test_ExacTest | OR | Gen_test_ExacTest | HWE_cases |

RENNES

| | | | | | | | | |
|---|---|---|---|---|---|---|---|---|
| SNP_A-1753080 | 4 | 55351790 | | | | | | |
| SNP_A-1753252 | 4 | 55352010 | | | | | | |
| SNP_A-1754613 | 4 | 55352994 | T | 0.065 | 0.018 | 1.3 | 0.023 | 0.91 |
| SNP_A-1655751 | 4 | 55374549 | | | | | | |
| SNP_A-1670843 | 4 | 55434793 | A | 0.041 | 0.013 | 1.6 | 0.033 | 0.58 |
| SNP_A-1712954 | 4 | 55451346 | | | | | | |

HUDDINGE

| | | | | | | | | |
|---|---|---|---|---|---|---|---|---|
| SNP_A-1753080 | 4 | 55351790 | | | | | | |
| SNP_A-1753252 | 4 | 55352010 | C | 0.019 | 0.043 | 2.2 | 0.04 | 1 |
| SNP_A-1754613 | 4 | 55352994 | | | | | | |
| SNP_A-1655751 | 4 | 55374549 | | | | | | |
| SNP_A-1670843 | 4 | 55434793 | A | 0.031 | 0.056 | 1.5 | 0.032 | 0.088 |
| SNP_A-1712954 | 4 | 55451346 | | | | | | |

SERACARE

| | | | | | | | | |
|---|---|---|---|---|---|---|---|---|
| SNP_A-1753080 | 4 | 55351790 | | | | | | |
| SNP_A-1753252 | 4 | 55352010 | | | | | | |
| SNP_A-1754613 | 4 | 55352994 | | | | | | |
| SNP_A-1655751 | 4 | 55374549 | A | 0.037 | 0.22 | 1.2 | 0.016 | 0.026 |
| SNP_A-1670843 | 4 | 55434793 | | | | | | |
| SNP_A-1712954 | 4 | 55451346 | | | | | | |

### Example 2

### c-Kit Enzyme Assay

The baculovirus donor vector pFbacG01 (GIIBCO) is used to generate a recombinant baculovirus that expresses the amino acid region amino acids 544-976 of the cytoplasmic kinase domains of human c-Kit. The coding sequences for the cytoplasmic domain of c-Kit is amplified by PCR from a human uterus c-DNA library (Clontech). The amplified DNA fragment and the pFbacG01 vector are made compatible for ligation by digestion with BamHI and EcoRI. Ligation of these DNA fragments results in the baculovirus donor plasmid c-Kit. The production of the viruses, the expression of proteins in Sf9 cells and the purification of the GST-fused proteins are performed as follows: Production of virus: Transfer vector (pFbacG01-c-Kit) containing the c-Kit kinase domain is transfected into the DH10Bac cell line (GIBCO) and the transfected cells are plated on selective agar plates. Colonies without insertion of the fusion sequence into the viral genome (carried by the bacteria) are blue. Single white colonies are picked and viral DNA (bacmid) is isolated from the bacteria by standard plasmid purification procedures. Sf9 or Sf21 cells (American Type Culture Collection) are then transfected in 25 cm² flasks with the viral DNA using Cellfectin reagent. Determination of small scale protein expression in Sf9 cells: Virus containing media is collected from the transfected cell culture and used for infection to increase its titre. Virus containing media obtained after two rounds of infection is used for large-scale protein expression. For large-scale protein expression 100 cm² round tissue culture plates are seeded with 5×10⁷ cells/plate and infected with 1 mL of virus-containing media (approx. 5 MOIs). After 3 days the cells are scraped off the plate and centrifuged at 500 rpm for 5 min. Cell pellets from 10-20, 100 cm² plates, are resuspended in 50 mL of ice-cold lysis buffer (25 mM Tris-HCl, pH 7.5, 2 mM EDTA, 1% NP-40, 1 mM DTT, 1 mM PMSF). The cells are stirred on ice for 15 min and then centrifuged at 5000 rpms for 20 min.

Purification of GST-tagged protein: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged protein is eluted by 10 applications (1 mL each) of 25 mM Tris-HCl, pH 7.5, 10 mM reduced-glutathione, 100 mM NaCl, 1 mM DTT, 10% Glycerol and stored at −70° C.

Kinase assay: Tyrosine protein kinase assays with purified GST-c-Kit are carried out in a final volume of 30 μL containing 200-1800 ng of enzyme protein (depending on the specific activity), 20 mM Tris-HCl, pH 7.6, 3 mM MnCl₂, 3 mM MgCl₂, 1 mM DTT, 10 μM Na₃VO₄, 5 μg/mL poly(Glu, Tyr) 4:1, 1% DMSO, 1.0 μM ATP and 0.1 μCi [γ³³P] ATP. The activity is assayed in the presence or absence of inhibitors, by measuring the incorporation of ³³P from [γ ³³P] ATP into the poly(Glu, Tyr) substrate. The assay (30 μL) is carried out in 96-well plates at ambient temperature for 20 min under conditions described below and terminated by the addition of 20 μL of 125 mM EDTA. Subsequently, 40 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4.times. on a shaker with 1.0% H₃PO₄ and once with ethanol. Membranes are counted after drying at ambient temperature, mounting in Packard TopCount 96-well frame, and addition of 10 μL/well of Microscint™ (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P ATP transferred from [γ³³P] ATP to the substrate protein per minute per mg of protein at 37° C.

Preferred c-kit inhibitors as used for the present invention exhibit, in the above-described assay, an IC₅₀ value between 50 and 2500 nM, more preferably between 250 and 2000 nM, and most preferably between 500 and 1250 nM.

### Example 3

### Combination Therapy

Utility of the c-kit inhibitors and the combinations treatments in treating demyelinating diseases, e.g. multiple sclerosis or Guillain-Barre syndrome as hereinabove specified, may be demonstrated in animal test methods, for example in accordance with the methods hereinafter described. The most widely used animal model for multiple sclerosis is Experimental Autoimmune Encephalomyelitis (EAE), based on shared histopathological and clinical features with the human disease:

The chronic EAE model in C57BY6 mice shares some common traits with the primary progressive (PP) or secondary progressive (SP) forms of MS. Mice are immunized in both flanks at day 0 and day 7 with 200 μg s.c. of myelin oligodendrocyte glycoprotein (MOG) in Complete Freund's Adjuvant (CFA) and followed by two injections (on day 0 and day 2) with 500 ng i.p. of B. pertussis toxin.

Groups are composed of 10 to 13 EAE mice. Clinical scores, overall health status, body weight and mortality are recorded daily. Starting from day 7 the animals are individually examined for the presence of paralysis by means of a clinical score: 0=no sign of disease, 1=tail paralysis, 2=tail paralysis+hindlimb weakness or partial hindlimb paralysis, 3=tail paralysis+complete hindlimb paralysis, 4=tail paralysis+hindlimb paralysis+weakness or partial paralysis of forelimbs, 5=moribund or dead.

Starting from day 10-12, most animals are becoming increasingly paralysed. The pathology is chronic and animals do not show signs of remissions after the first clinical signs of disabilities, and during the following 28 to 30 days of observation.

Therapeutic treatments are started at the onset of the disease, thus once the disease is already established but still progressing and continued for 28 to 30 days. Subcutaneous daily treatment with mIFNβ (Serono Pharmaceutical Research Institute, Geneva) at the dose of 20,000 U/mouse shows beneficial effects on clinical output by significantly reducing the severity of the disease from complete hindlimb to partial hindlimb paralysis. Combination therapy of compounds with mIFNβ can be achieved by daily double treatment with either suboptimal (5000 U/mouse) or optimal mIFNβ dose. Control vehicle-treated EAE groups following the same administration routes are included in experiments.

1-29. (canceled)

30. A composition comprising a c-kit inhibitor and a neuroactive compound.

31. The composition according to claim 30, wherein the c-kit inhibitor is a selective c-kit inhibitor.

32. The composition according to claim 31, wherein the c-kit inhibitor is selected from imatinib, ZK-222584, CT-53518 or semaxinib.

33. The composition according to claim 30, wherein the neuroactive compound is selected from neuro-protective agents, immunosuppressive drugs, immunomodulatory drugs, corticosteroids, cytokines, or combinations thereof.

34. The composition according to claim 33, wherein the neuroactive compound is an interferon.

35. The composition according to claim 34, wherein said interferon is a beta-interferon.

36. The composition according to claim 35, wherein said beta-interferon is human interferon beta-1a.

37. The composition according to claim 30, wherein the c-kit inhibitor is selected from imatinib, ZK-222584, CT-53518 or semaxinib and the neuroactive compound is selected from neuro-protective agents, immunosuppressive drugs, immunomodulatory drugs, corticosteroids and cytokines, or combinations thereof.

38. The composition according to claim 37, wherein the netroactive compound is an interferon.

39. The composition according to claim 38, wherein said interferon is a beta-interferon.

40. The composition according to claim 39, wherein said beta-interferon is human interferon beta-1a.

41. A method of treating a subject having multiple sclerosis comprising the administration of a composition comprising a c-kit inhibitor and a neuroactive compound to said subject.

42. The method according to claim 41, wherein the c-kit inhibitor and neuroactive compound are administered simultaneously.

43. The method according to claim 41, wherein the c-kit inhibitor and neuroactive compound are administered sequentially.

44. The method according to claim 41, wherein the c-kit inhibitor and neuroactive compound are administered repeatedly.

45. The method according to claim 41, wherein said neuroactive compound is an interferon that is administered daily or every other day.

46. The method according to claim 41, wherein said neuroactive compound is an interferon that is administered twice or three times a week.

47. The method according to claim 41, wherein said neuroactive compound is an interferon that is administered at a dosage of about 1 to 50 μg per person, 1 to three times a week.

48. The method according to claim 41, wherein said neuroactive agent is administered by subcutaneous injection(s).

49. The method according to claim 41, wherein the subject has a susceptibility alteration in a c-kit gene or polypeptide.

50. A method of treating a subject with a disease selected from phenylketonuria and other aminoacidurias; Tay-Sachs disease; Niemann-Pick disease; Gaucher's disease; Hurler's syndrome; Krabbe's disease; Acute disseminated encephalomyelitis; Acute inflammatory peripheral neuropathies; Guillain-Barre syndrome; adrenoleukodystrophy; adrenomyeloneuropathy; progressive multifocal leukoencephalopathy (PML); acute disseminated encephalomyelitis (ADEM); Leber's hereditary optic atrophy; HTLV-associated myelopathy; or Pelizaeus-Merzbacher disease comprising the administration of a composition according to claim 30 to said subject.

51. A method of detecting the presence of or predisposition to multiple sclerosis comprising detecting in vitro or ex vivo the presence of a susceptibility alteration in a c-kit gene or polypeptide in a sample from the subject, the presence of such an alteration being indicative of the presence of or predisposition to multiple sclerosis.

52. The method according to claim 51, wherein the susceptibility alteration is a single nucleotide polymorphism (SNP) selected from those listed in Tables 2 and 3.

53. The method according to claim 52, wherein the susceptibility alteration is detected by sequencing, selective hybridisation and/or amplification.

54. A method of assessing the response or responsiveness of a subject to a treatment for multiple sclerosis comprising detecting in vitro or ex vivo the presence of a susceptibility alteration in a c-kit gene or polypeptide in a sample from the subject, the presence of such an alteration being indicative of a responder subject.

55. The method according to claim 54, wherein the susceptibility alteration is a single nucleotide polymorphism (SNP) selected from those listed in Tables 2 and 3.

56. The method according to claim 55, wherein the susceptibility alteration is detected by sequencing, selective hybridisation and/or amplification.
